(19) United States
(12) Patent Application Publication
(10) Pub. No.: US 2005/0118194 A1
(43) Pub. Date: Jun. 2, 2005

(54) Oral vaccine, method for its preparation and use thereof

(76) Inventors: Yoke Min Sin, Singapore (SG); Hsiao Chuin Teh, Singapore (SG); Sze Yun Lim, Singapore (SG)

Correspondence Address: HARNESS, DICKEY & PIERCE, P.L.C., P.O. BOX 828, BLOOMFIELD HILLS, MI, 48303, US

(21) Appl. No.: 10/725,188

(22) Filed: Dec. 1, 2003

(51) Int. Cl. A61K 39/00; A61K 39/295; A61K 35/78

(52) U.S. Cl. 424/201.1; 424/442; 424/776

(57) ABSTRACT

A composition for oral immunization comprising recombinant proteins AHMA and FP that afford protection from bacteria and protozoan ectoparasites respectively is disclosed. Also disclosed are compositions that have in addition, inactivated viruses and killed bacteria affording a wider spectrum of protection against infections that predominantly afflict aquatic species. Method of making these compositions and administering them either directly or by incorporating them into feed, as also the use of such compositions for treating animals in need of such treatment are also disclosed.

### TECHNICAL FIELD

The invention pertains to vaccines against infectious pathogens and method of producing them. More particularly, it pertains to fish vaccines.

### BACKGROUND

Aquaculture has gone through major changes, ranging from small-scale homestead-level activities to large-scale commercial farming. Over the past three decades the sector has expanded, diversified, intensified and advanced technologically to contribute significantly to aquatic food production. It significantly contributes to food security, poverty alleviation and social well-being in many countries. The contributions of aquaculture to trade have also increased over recent decades and its share in the generation of income and employment has also increased significantly. Commercial aquaculture requires maintenance of high density of fishes. The likelihood of serious economic losses therefore is very high when the cultured fish population becomes infected by pathogens. There is widespread occurrence of epizootics in fish farms caused by a variety of pathogens, including protozoans, bacteria and viruses.

Traditionally, introducing chemotherapeutic agents such as sulfa drugs or oxytetracyclines has been the method of choice for treating bacterial infections. This method has several inherent deficiencies. Many bacterial strains have been known to develop resistance because of unregulated use of such a strategy. The process is very expensive and cumbersome. Besides the environmental problems created, the strategy does not help in treating diseases of viral etiology, which are equally prevalent. The preference for immunogens or vaccines to treat fish disease has therefore gained prominence in recent years.

Efforts have been made to develop vaccines against selected fish pathogens. Thus, a vaccine has been developed against Y. ruckerii (Tebbit et al., Developments in Biological Standardization, Vol. 49, International Symposium on Fish Biologics: Serodiagnostics and Vaccines, W. Heunessen and D. P. Anderson (eds.), 1981, pp. 395-402), and V. anguillarum (Amend and Johnson, Developments in Biological Standardization, Vol. 49, International Symposium on Fish Biologics: Serodiagnostics and Vaccines, W. Heunessen and D. P. Anderson (eds.), 1981, pp. 403-418; Agius et al, J. Fish Dis. 6, 1983, pp. 129-134). These vaccines are based on formalin-killed virulent bacteria. The efficacy of these vaccines has been tested and it has been shown that the route of administration of the vaccines plays an important part in the strength of the resulting immune response (Kawano et al., Bull. Jpn. Soc. Sci. Fish. 50, 1984, pp. 771-774; Ward et al, in Fish Immunology, M. J. Manning and M. F. Tatner (eds.), 1985, pp. 221-229). Further, a vaccine comprising chloroform-inactivated whole cells, soluble antigen and combined whole cell and soluble antigen of an avirulent strain of Aeromonas salmonicida has been shown to protect fish against furunculosis (Cipriano et al., J. World Maricul. Soc., 1983, 14, 201-211).

Aeromonas hydrophila is a gram-negative bacterium that infects a wide range of hosts including mammals, birds, reptiles and amphibians (Popoff, M. Aeromonas. In: Bergy's Manual of Systematic Bacteriology, N. R. Krieg (ed), Williams & Wilkins, Baltimore, Md. 1984, vol.1, pp.545-548), but it is most well-known as a pathogen of aquatic animals such as fish. It causes motile aeromonad septicemia (MAS), which results in great economic losses in freshwater fish farming. Antibiotics are often used for prevention and treatment of MAS (Stevenson, R W M. “Vaccination against Aeromonas hydrophila” In: Fish Vaccination. Ellis, A E (ed), Academic Press, London, 1988, pp 112-123). However, extensive use of antibiotics has serious drawback of increasing plasmid-coding antibiotic resistance in A. hydrophila. Due to the antigenic diversity of A. hydrophila strains, it has been difficult to develop a useful vaccine and accordingly there are no effective vaccines, currently known or commercially available for protection against wide range of different virulent strains of A. hydrophila.

Similarly, protozoan ectoparasites take a toll on fish population. For example, a ciliated protozoan, Icthyophthirus multifiliis causes white spot or ïch, especially in ornamental fish. It was established that immunity could be conferred in laboratories even by parasite exposure (Hines and Spira 1974). However, there are no effective vaccines commercially available. Besides, there are a number of bacterial and viral infections that are prevalent such as Aeromonas hydrophila, vibrios, reovirus and nervous necrosis virus that pose serious commercial threat to the aquaculture industry.

The route of delivering a vaccine is an important factor for successful immunization. Generally, intra-peritoneal and intramuscular immunizations with immunogens have been shown to generate long-lasting and protective immunity in immunized animals. However, besides the problems of handling very small or large fishes and administering adequate dosages to them, the procedure can be very stressful to the recipient. The other method that is commonly practiced in this field is delivering a high concentration of an immunogen in the water for uptake by the animals for example the immersion or bathing method. Besides being a labour-intensive process, the procedure is also wasteful! It is limited by the weight of fish that can be immunized per unit volume of vaccine.

A need thus clearly exists to develop vaccines that can elicit effective immunological protection against a broad spectrum of pathogens in a cost-effective and labour-efficient manner.

### SUMMARY

It is thus an object of the instant invention to provide a composition that when orally administered, can protect animals against ubiquitous bacterial and viral infections as well as some of the protozoan ectoparasites particularly prevalent in the aquatic environment.

An embodiment of the invention provides a composition comprising recombinant protein major adhesin protein of Aeromonas hydrophila (AHMA).

Another embodiment of the invention provides a composition comprising two recombinant proteins namely, AHMA and immobilization antigen repeat I of Ichthyophthirius multifiliis (Fusion protein or FP hereafter), to constitute a multicomponent vaccine affording protection against common aquatic infections.

In accordance with one aspect of the instant invention, there is also provided a composition wherein killed bacteria selected from a group consisting of Shiwanella putrefaciens, Pseudomonas florescens, Vibrio alginolyticus and Flexinobactor columnaris or their respective antigens are included along with recombinant AHMA and FP.

Another aspect provides a composition wherein inactivated guppy reovirus (GPV) and guppy nervous necrosis virus (GNNV) or their coat proteins are further included to the oral vaccine having AHMA, FP and bacterial antigens as described above.

In accordance with an aspect of the invention, there is provided a vaccine that is amenable to mixing with feed to facilitate oral delivery of antigens.

In another aspect of the present invention, there is provided a method of delivering a vaccine by emulsifying the immunogens in a water-in-oil emulsion.

Another aspect of the invention provides a method of entrapping physico-chemically-sensitive biological molecules such as polypeptides for safe delivery through the gut without their being readily degraded by the gastric enzymes.

According to yet another aspect of the invention, a method is provided for sustained release of a vaccine composition in the immunized animal whereby the vaccine is not degraded all at once but does so over a longer duration, giving the immune system a longer exposure to the antigens of the composition than is ordinarily possible.

In accordance with another aspect of the present invention, there is also provided a particulate vaccine wherein the emulsified immunogens are adsorbed onto a binding agent.

In accordance with yet another aspect of the invention, there is provided a vaccine delivery mode which is non-traumatic, safe and effective. Oral immunization has been associated with a sustained and longer-lasting immunological memory and the instant invention attempts to achieve that objective.

These and other advantages of the present invention will become apparent upon review of the following detailed description of the invention and the claims.

### BRIEF DESCRIPTION OF THE DRAWINGS

FIG. 1 is a histogram comparing the protective effect of the multicomponent emulsion vaccine and the non-emulsion vaccine against A. hydrophila challenge.

FIG. 2 is a graph comparing the protection conferred by the vaccine against viral challenge, when administered orally and by the immersion method.

### DEFINITIONS

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

For the purposes of the present invention, the phrase “elicit(s) an immune response” refers to the ability of a polypeptide or immuno-interactive fragment or variant derivative, or a bacterial or a protozoan or viral molecule of the invention to produce an immune response in an animal to which it is administered, including the production of antibodies and cellular immunity components.

By “expression vector” is meant any autonomous genetic element capable of directing the synthesis of a protein encoded by the vector. Such expression vectors are known to practitioners in the art.

As used herein, the term “function” refers to a biological, enzymatic, or therapeutic function.

“Homology” refers to the percentage number of amino acids that are identical or constitute conservative substitutions. Homology may be determined using sequence comparison programs.

By “immunologically effective amount” is meant the administration to an animal of an amount of a protein, polypeptide, immuno-interactive fragment, variant or derivative, bacterial, protozoan or viral molecule of the invention, either in a single dose or as part of a series, that is effective for eliciting an immune response against that protein, polypeptide, immuno-int ractive fragment, variant or derivative or against a bacterium, protozoan or virus comprising said protein, polypeptide, immuno-interactive fragment, variant or derivative or surface molecule. The effective amount will vary depending upon the taxonomic group of animal to be treated, the capacity of the animal's immune system to elicit an immune response (inclusive of a humoral and/or a cellular immune response), and the formulation of the vaccine. It is expected that the amount will fall in a relatively broad range that can be determined through routine trials.

By “isolated” is meant material that is substantially or essentially free from components that normally accompany it in its native state. e.g., a DNA fragment which has been removed from the sequences which are normally adjacent to the fragment.

By “pharmaceutically-acceptable carrier” is meant a solid or liquid filler, diluent or encapsulating substance that can be safely used in topical or systemic administration to a fish.

By “polypeptide” is meant a molecule composed of amino acids that may be derived from natural sources, or artificially synthesized such as by using a peptide synthesizer.

The term “polypeptide derivative” refers to polypeptides in which one or more amino acids have been replaced by different amino acids and which retains the function or activity of the polypeptide. It is well understood in the art that some amino acids may be changed to others with broadly similar properties without changing the nature of the function or activity of the polypeptide (conservative substitutions) as described hereinafter. The term “recombinant polynucleotide” or “synthetic polynucleotide” refers to a polynucleotide formed in vitro by the manipulation of nucleic acid into a form not normally found in nature. For example, the recombinant or synthetic polynucleotide may be in the form of an expression vector. Generally, such expression vectors indude transcriptional and translational regulatory nucleic acid operably linked to the nucleotide sequence.

By “recombinant polypeptide” is meant a polypeptide made using recombinant techniques, i.e., through the expression of a recombinant or synthetic polynucleotide.

By “immunogen” or “antigen” is meant a molecule that when administered into the body of a recipient animal, elicits an immune response.

By “emulsion” is meant a mixture of two immiscible liquids wherein one is dispersed in the other in minute droplets.

By “oral administration” is meant administering the vaccine or feed-stuff comprising the vaccine to the oral cavity of individual recipients by any suitable means including mechanical or manual administration.

### DETAILED DESCRIPTION

One embodiment of the instant invention provides an oral vaccine comprising at least one recombinant protein AHMA. In one preferred embodiment of the invention, the composition comprises two recombinant proteins namely, recombinant AHMA and recombinant FP dissolved in an emulsion. The cloning and expression of recombinant AHMA has been fully described in the U.S. patent application Ser. No.10/220,986 to Sin et al. filed on 7^{th} Mar., 2001 entitled “Therapeutic and Prophylactic Agents Derived from Aeromonas hydrophila Bacterial Surface Proteins”, the contents of which in its entirety are incorporated by reference herein. Cloning and expression of recombinant FP has been fully described in U.S. patent application Ser. No. 09/196,161 to Sin et al. filed on 20^{th} Nov., 1998 entitled “Recombinant Vaccine Against Infectious Disease in Fish”, the contents of which in its entirety are incorporated by reference herein. Other embodiments may incorporate other recombinant coat proteins from pathogens to enhance the protection of the vaccine against a wider spectrum of infections. Thus, an embodiment of the invention comprises recombinant proteins AHMA and FP along with killed bacteria selected from the group consisting of Shiwanella putrefaciens, Pseudomonas florescens, Vibrio alginolyticus and Flexinobactor columnaris. Another embodiment of the invention includes besides the recombinant proteins and killed bacteria mentioned above, inactivated viruses from the group consisting of guppy reovirus and guppy nervous necrosis virus.

In work leading up to the present invention, a 43-kDa outer membrane protein of A. hydrophila strain PPD 134/91 was identified as an important adhesin molecule (Lee, et al., 1997, Journal of Fish Diseases 20: 169-175). N-terminal sequence analysis of this protein revealed a 20-residue sequence with substantial homology to the 39 kDa outer membrane protein, Omp II, of A. hydrophila Ah 65 isolated from the rainbow trout. The sequence information enabled the cloning and expression of the recombinant protein AHMA, which is the subject matter of U.S. patent application Ser. No. 10/220,986 referenced above. It is possible to engineer mutants effecting substitutions in the amino acid positions, or deletions or additions with a proviso however, that the mutation(s) or addition/deletion do not critically reduce the immunogenicity of the molecule. The recombinant AHMA may be administered alone as an oral vaccine. It may also be used as a component along with other protective antigens in a multicomponent vaccine. Recombinant AHMA may be used to elicit protection against other infections to which the antibodies it generates are cross-reactive. Thus, protection against the bacterial genus of Aeromonas, Vibrio and Edwardsiella may be afforded by the recombinant protein. Any suitable expression system that would express the recombinant proteins in a near-native conformation may be employed. Preferentially, E. coli expression system wherein the recombinant product can be sequestered in the periplasmic space, may be adopted for large-scale preparation of recombinant AHMA antigen. Expressing the recombinant AHMA as a fusion protein along with another polypeptide such as glutathione S-transferase (GST) is also envisaged. This may increase the efficiency of the recovery process.

Similarly, recombinant FP may be made according to the teaching in U.S. patent application Ser. No. 09/196,161. It may preferentially be expressed as a fusion protein along with GST, or such other protein to facilitate recovery and purification operations. A proteolytic site engineered between the two protein sequences can enhance separation efficiency of FP from GST. The immobilization antigen of Icthyophthirius multifiliis was cloned and expressed in E. coli as a fusion protein with GST, the cloning and expression of which is the subject matter of U.S. patent application Ser. No. 09/196,161 referenced above. FP affords protection against ectoparasitic ciliated protozoans. Recombinant FP may therefore be useful as a component of a multivalent vaccine formulation.

The invention also relates to the use of AHMA protein either alone or in combination with FP recombinant protein, its fragments or variants modified using conventional molecular biology techniques, in order to improve the yield, recoverability, stability, solubility or immunogenicity. It is possible to use mutated polypeptides or conservative substituents wherein amino acids are changed without any loss of immunogenicity. Cloning of polynucleotides encoding antigenic determinants of AHMA, FP, or viruses either individually or as fusion cassettes into suitable expression vectors and cell lines that would provide proteins bearing immunogenic properties substantially similar to the native molecules, is also envisaged.

It may be advantageous to add inactivated viruses or their antigenic components to the vaccine as it may be relevant to extend the protection of the vaccine to other species of viral pathogens in the aquatic milieu. These may be crudely prepared from the viral source, for example, by lysing the cell-lines hosting the virus and harvesting virus from the supernatant. The virus may be killed or inactivated by any method known to persons of ordinary skill in the art. These methods may include irradiation or heat-shock or by chemical treatment using formaldehyde, glutaraldehyde, beta-propiolactone or ethyleneimine. Viral antigens may be expressed by recombinant methods and only the purified and antigenically relevant epitopes may be incorporated as component of the vaccine preparation. For example, the guppy reovirus or the guppy nervous necrosis virus coat proteins produced by recombinant DNA techniques can be incorporated into the oral vaccine.

While recombinant AHMA may generate antibodies that cross-protect against other bacterial species, such as of Aeromonas, Vibrio and Edwardsiella, it may be desirable to add other bacterial antigens to the oral vaccine. Thus in one embodiment of the invention, four killed bacteria namely, Shiwanella putrefaciens, Pseudomonas florescens, Vibrio alginolyticus and Flexinobactor columnaris are incorporated. These bacterial infections are common in fish. However, caution must be exercised while selecting bacterial antigens for incorporation namely, that they must not cross-react with AHMA antibodies, as recombinant AHMA is one component of the oral vaccine. These selected bacteria may be inactivated by any method known to those skilled in the art, including irradiation, heat-inactivation or chemical treatment. Their antigenic proteins may also be made by recombinant methods for incorporation into the multicomponent oral vaccine.

The recombinant proteins may be dissolved in water or saline to make the aqueous phase prior to mixing with organic oil for making an emulsion. Any metabolizable oil especially vegetable oil may be used to make the emulsion. Generally, any organic oil if metabolizable and non-toxic, may be used to make the emulsion. Since the vaccine is intended for oral administration, preferably organic oil may be used. These may be vegetable oil, animal oil or fish oil or any synthetically prepared oil that can be metabolized by the recipient. These may be selected from amongst peanut, soybean, olive, palm oil, coconut, sunflower, cotton-seed, safflower, sesame etc. Oil from grains may also be used. In the instant invention, palm oil was found to be eminently suitable for making a good emulsion for the vaccine.

Suitable binding agents may be added to the emulsified vaccine preparation to give it particulate consistency. If particulate composition is desired, the emulsion may be mixed with granular feed composition. For immunizing fish for example, the vaccine could be mixed with feed such as eel feed as was done in the instant invention. The invention provides oral vaccine of granular composition. Also, other particulate matter that are biologically inert may be used to render the vaccine into a particulate form. This may include high viscosity carboxymethylcellulose. Other suitable materials may include powdered animal feeds and powdered edible inorganic material. Colouring agents or food dyes may be used to make the vaccine composition attractive to the intended recipient

The vaccine may be mixed with a binding agent and extruded into solid pellets. The vaccine may also be added to animal feed as paste and administered orally to intended recipients. The oral vaccine may also include suitable carrier or diluent, stabilizers to prevent the emulsified vaccine from degrading on storage. Mild non-ionic surfactants may be incorporated into the formulation to obtain uniform particulate size.

The oral vaccine may be administered either by incorporating in feed-stuff for the recipient during manufacture of the feed itself. Accordingly, the invention also provides an animal feedstuff comprising an oral vaccine composition as described above. Alternatively, the vaccine may be simply added to the feed at the time the feed is fed to the animal by sprinkling the vaccine on the feed.

Also envisaged are the use, of adjuvants, plasticizers, pharmaceutical excipients, other soluble antigens, diluents, carriers, stabilisers, binders, lubricants, glidants, colouring agents, flavours and combinations thereof, with the vaccine.

The oral vaccine may be administered to any animal potentially at risk of infection by Aeromonas hydrophila. These may include fishes, amphibians, reptiles, birds and mammals. A. hydrophila is also an opportunistic human pathogen. Similarly, the vaccine is suited to immunize against Edwardsiella and Vibrio bacteria too. Aquatic animals and primarily fish are more at risk. The vaccine may be used effectively on all fishes. However, economic losses are greater due to this infection amongst ornamental fishes. The vaccine is efficacious in protecting many ornamental fish from infection, including the guppy, goldfish and the blue gourami, as described in the following experiments. The multicomponent oral vaccine incorporating I. multifliis, killed bacteria and inactivated virus can afford protection to fish from multiple diseases of the aquatic environment including white spot disease,

The oral vaccine is made by expressing recombinant protein AHMA or its immunogenic fragments as described elsewhere and emulsifying the recombinant protein in a water-in-oil emulsion. The proportion of oil and water may be in the ratio of 2:1. Preferably they may be equal proportions. In the experimental study, excellent results were obtained when 2.5 ml water or saline and 5 ml palm oil was used to make the emulsion. The emulsion may be administered as such orally. Or it may be rendered into a particulate form by the addition of edible binding agents to the emulsion. Conservative substitutions that do not drastically reduce immunogenicity or protective response may be used for the vaccine. Other components such as recombinant FP, viral proteins and killed bacteria may be separately combined to the recombinant AHMA-emulsion. These together may be gently mixed into particulate edible feed to give the vaccine a particulate consistency. These particulate material may include animal feed, or biologically inert material such as high viscosity carboxymethyl cellulose. The vaccine may also be sprayed in its emulsified form onto feed for easy oral administration.

The dosage of components of the vaccine is made in accordance with the body weight of the intended subject so as to provide an immunologically sufficient amount to elicit protective response. Thus dosage of AHMA in the vaccine may range between 7 and 150 μg/gm body weight. A more preferable amount would be 15-20 μg/gm, a most preferred amount would be about 17 μg/gm body weight. Similarly the components may also be employed at immunologically effective dosage. Thus, an immunologically effective amount of recombinant FP may range between 7 and 150 μg/gm body weight, a more preferable dose range may be 15 and 20 μg/gm and the most preferable dose may be 17 μg/gm body weight of the immunized subject. Preferred amounts of inactivated virus or equivalent amounts of viral proteins for guppy reovirus and guppy nervous necrosis virus may range between 10³ to 10⁶ viral particles per unit dose of the vaccine. The most preferred amounts to elicit a protective response may be 10⁵ viral particles of each virus per dose of the vaccine. Similarly, killed bacteria or bacterial protein components to be used in the vaccine including S. putrefaciens, P. florescence, V. alginolyticus and F. columnaris may have a range of 2.5×10⁵ to 2.5×10⁷ cfu of each of the bacterium. The most preferred amount may be 2.5×10⁶ cfu of each bacterium or its equivalent coat protein per unit dose of the vaccine.

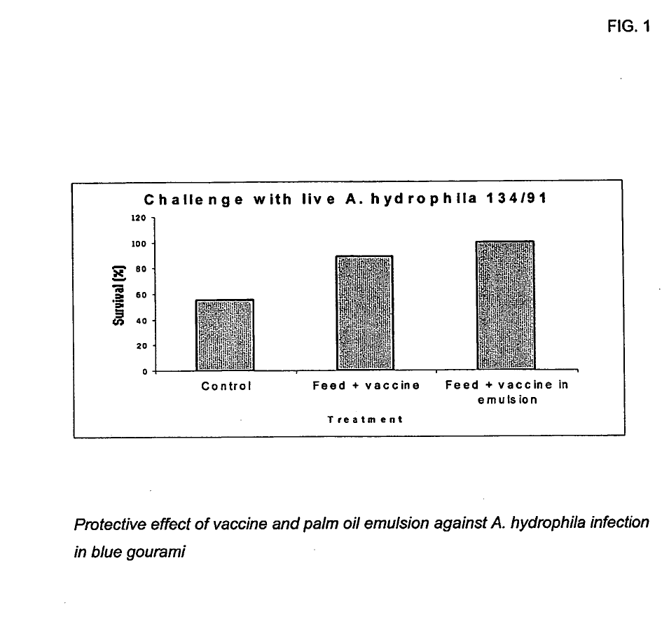

Referring now to the figures, FIG. 1 is a histogram comparing the protective effect of the multicomponent vaccine made as an emulsion and mixed with feed and the vaccine administered by directly mixing with feed against A. hydrophila challenge. Three groups of blue gourami fish were administered the multicomponent vaccine either in emulsified form mixed with feed or just added to the feed in a non-emulsion form. Controls were given just the feed alone. Post-immunization challenge with A. hydrophila show that while there was 50% survival amongst controls, immunization with either form of the vaccine did significantly increase protection in the experimental group. It was observed that the vaccine administered to the fish in the emulsion-form mixed with feed, conferred on the recipients a higher survival rate in comparison to recipients who were administered the vaccine just mixed with feed.

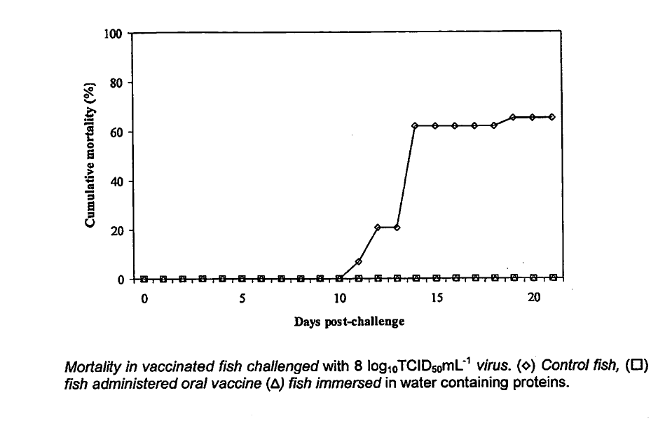

FIG. 2 depicts a graph comparing the protection conferred by the oral vaccine against viral challenge. The vaccine was orally administered and the protein cocktail was administered by immersion method. Fish immunized with the multicomponent oral vaccine either orally or by immersion survive the challenge and so do the fish immersed in the protein cocktail. Fish from the control group receiving feed alone, begin succumbing to the infection on day 10 post-challenge and about 60% die by the 20^{th} day post-challenge.

The invention will be better understood from the reading of the following non-limiting examples which are provided only for illustrative purposes.

### EXAMPLE I

Expression and Purification of AHMA

The gene construct pQE-AHMA, encoding the AHMA 43 kDa polypeptide was obtained from Fang Haoming, National University of Singapore and transformed into E. coli (M15, QIAGEN). The 3 hour culture of E coli harbouring pQE-AHMA was diluted to 1:20 in fresh LB medium containing 100 ug/ml ampicillin and 15 ug/ml kanamycin and grown at 37° C. with vigorous shaking. IPTG was added to a final concentration of 1 mM when OD₆₀₀ of the bacterial culture reached 0.5. Three hours after the addition of IPTG, bacteria were harvested by centrifugation at 480 g for 10 min at 4° C.

The AHMA recombinant protein was isolated using the following method: Briefly, for every 500 ml of bacterial culture, the harvested bacteria were re-suspended in 10 ml of FP lysis buffer (50 mM Tris, 0.1 M NaCl, 1 mM EDTA pH 8.0). The tube was then immersed in ice and the cells were lysed using a probe sonicator with a 5-mm-diameter probe for 6×30 sec. The lysate was centrifuged at 8000 g at 4° C. for 1 hr. The resultant pellet was re-suspended in AHMA lysis buffer (8 M Urea, 100 mM NaH₂PO₄, 10 mM Tri-HCl, pH 8.0). After 30 min of shaking in AHMA lysis buffer, the cell suspension was centrifuged at 14,000 g at 4° C. for 1 hour. The resultant supernatant contains AHMA. The pooled AHMA was analyzed by SDS-PAGE and its concentration was measured using BIORAD protein Assay (BIORAD). Urea was removed from the AHMA by gradual dialysis against buffers (Urea, 100 mM NaH₂PO₄, 10 mM Tri-HCl, pH 7.4) containing different concentrations of urea. Dialysis with buffer F (1 M Urea, 100 mM NaH₂PO₄, 10 mM Tri-HCl, pH 7.4) was followed by PBS (pH 7.4). The AHMA in PBS was freeze-dried and stored until use.

### EXAMPLE II

Expression and Purification of GST Fusion Protein (FP)

The plasmid for GST-FP, pGST-iAg obtained from Stratagene was transformed into E. coli (M15, QIAGEN). A 3-hour culture of E. coil harboring pGST-iAg was diluted to 1:20 in fresh LB medium containing 100 ug/ml ampicillin and 15 ug/ml kanamycin and grown at 37° C. with vigorous shaking. Isopropyl 1-thio-β-D-galactoside (IPTG) was added to a final concentration of 1 mM when OD₆₀₀ reached 0.5. Bacteria were harvested 3 hours after the addition of IPTG by centrifugation at 2000 g for 10 min.

FP was purified by Glutathione Sepharose 4B Beads (PHARMACIA) as described in He J. Y., Yin Z., Xu G. L., Gong Z. Y., Lam T. J., Sin Y. M. (1997) Protection of goldfish against Ichthyophthirius multifiliis by immunization with a recombinant vaccine. Aquaculture. 158, 1-10. Briefly, the collected pellet for every 1 L of bacterial culture was re-suspended in 10 ml of FP lysis buffer (50 mM Tris, 0.1 M NaCl, 1 mM EDTA, pH 8.0), to which lysozyme was added to a final concentration of 5 mg/ml. After shaking at room temperature for 5 min, bacteria were lysed with 1% Triton-100. The resulting suspension was shaken for a minimum of 30 min. The lysate was cleared by centrifugation at 14000 g for 1 hour at 4° C.

The supernatant was incubated with 1 ml of 50% slurry of Glutathione Sepharose 4B beads (Pharmacia) at room temperature for 1 hour with gentle agitation. The beads were then washed thrice in 10× bed volume of FP PBS (150 mM NaCl, 16 mM Na₂HPO₄, 4 mM NaH₂PO₄). FP was eluted from beads with 15 mM reduced glutathione. The collected FP was analyzed by SDS-PAGE and concentration was measured using BIORAD protein Assay (BIORAD). Glutathione was removed by dialyzing against phosphate buffered saline, PBS (130 mM NaCl, 3 mM NaH₂PO_{4,} 7 mM Na₂HPO₄ pH 7.4) and the FP was freeze-dried for storage until its use in the vaccine.

### EXAMPLE III

Preparation of Crude Viral Antigen(s)

Blue gill fry cell line (BF-2, ATCC CCL91), was used for the primary isolation and propagation of the guppy virus (GPV) at 25° C. Guppy nervous necrosis virus (GNNV) was cultured in sea bass (SB) cell line derived from Asian sea bass larvae.

Virus infectivity was assayed according to method of Payment and Trudel (1993). The infected cells were incubated at 25° C. and monitored daily for cytopathic effect (CPE) in the wells. Once the CPE had stopped progressing, the titre was determined using the method of Reed and Müench (1938), that evaluates an endpoint where 50% of the cell cultures are infected. A formula that takes into account the accumulated percentage of infected cultures was used to calculate tissue culture infectious dose (TCID₅₀).

To kill GPV and GNNV, 0.1% formalin was added to the cell culture supematant and remaining cells that were harvested from the flask wherein massive CPE had occurred. The formalin treated culture was left at 4° C. for 17 days. After 17 days, 35% sodium thiosulphate (volume equivalent to ⅓ the volume of formalin) was added. The virus was then dialyzed 4 times In PBS, with 12 hourly changes of PBS. SDS-PAGE was carried out to determine presence of any infective virus and the vaccine was inoculated onto BF-2 or SB monolayer cells respectively to determine its virulence or toxicity.

### EXAMPLE IV

Preparation of Bacterial Antigen(s)

Four strains of bacteria (Shiwanella putrefaciens, Pseudomonas florescens, Vibrio alginolyticus and Flexibactor columnaris) were grown separately. S. putrefaciens, P. florescens and V. alginolyticus were cultured in TSB while the F. columnaris was cultured in Ordal culture media (0.2% tryptone, 0.05% yeast, 0.3% gelatin).

Briefly, fresh medium was inoculated from an overnight bacterial culture and grown at 25° C. with vigorous shaking for about 3 hours till OD₅₄₀ reaches 0.5. Samples were plated onto TSB Agar plate or Ordal agar plate (1.5% agar into Ordal medium) respectively to calculate the CFU values. The bacterial culture was pelleted by centrifugation at 2,000 g for 15 min and washed once with PBS. The washed bacterial pellet was re-suspended in PBS and formalin was added to a final concentration of 0.4% v/v of the original bacterial culture volume. After a minimum of 4 days, the formalin-killed bacteria were pelleted and washed twice with PBS. Samples were plated to ensure total killing. The bacterial pellets were frozen and stored till its later use in the vaccine.

### EXAMPLE V

Preparation of Oral Vaccine

The various embodiments of the oral vaccine were prepared employing the following dosages for every batch of 100 fish:
(a) 0.7 mg AHMA for the oral vaccine comprising recombinant AHMA alone, (b) 0.7 mg AHMA+0.7 mg recombinant FP for the rAHMA-FP vaccine, (c) 0.7 mg AHMA+0.7 mg recombinant FP and 2.5×10⁶ cfu of each or all the four bacteria, Flexibacter columnaris, Pseudomonas florescens, Shiwanella putrefaciens and Vibrio alginolyticus for the oral vaccine that also included bacterial components and (d) 0.7 mg AHMA+0.7 mg recombinant FP, 2.5×10⁶ cfu of each or all the four bacteria, Flexibacter columnaris, Pseudomonas florescens, Shiwanella putrefaciens and Vibrio alginolyticus and 10⁵ viral particles of GPV and GNNV for the vaccine having viral antigens in addition. For the multi-component vaccine having AHMA, FP, the bacterial and viral antigens, all the 8 components were mixed in a total volume of 0.25 ml water and 0.5 ml palm oil. The mixture was vigorously stirred till it emulsified before being folded into 0.5 g of powdered commercial eel feed. The other embodiments were also prepared in a similar manner using the respective amounts of antigen indicated.

### EXAMPLE VI

Immunization of Blue Gourami with Recombinant Adhesin

Recombinant protein obtained from the pQE-AHMA transformed E. coli was used to immunize Blue gourami. Immunized animals were challenged with different strains of A. hydrophila, V. anguillarum and E. tarda. The following table shows the extent of protection afforded against these infections to immunized animals.

TABLE 1
Extent of protection in Blue gourami immunized with rAHMA vaccine

| Bacterial | | | Total | | | |
|---|---|---|---|---|---|---|
| strains | Dose | | fish | Dead | Survival | RPS^{b} |
| for challenge | (cells/ml) | Group^{a} | used | Fish | (%) | (%) |
| A. hydrophila | 6.0 × 10⁵ | Immune | 20 | 1 | 95 | 87.5** |
| PPD 134/91 | | Control | 20 | 8 | 60 | |
| A. hydrophila | 6.1 × 10⁵ | Immune | 20 | 3 | 85 | 70.0** |
| PPD 70/91 | | Control | 20 | 10 | 50 | |
| A. hydrophila | 4.45 × 10⁵ | Immune | 20 | 5 | 75 | 28.6 |
| L31 | | Control | 20 | 7 | 65 | |
| V. anguillarum | 1 × 10⁶ | Immune | 20 | 10 | 50 | 44.4* |
| 01/10/93(2) | | Control | 20 | 18 | 10 | |
| E. tarda | 3.28 × 10⁶ | Immune | 20 | 5 | 75 | 44.4 |
| PPD 130/91 | | Control | 20 | 9 | 55 | |

^{a}Duplicate group of 10 fish each. For immune group, fish were injected with 15 μg of recombinant adhesin in FCA; Fish in control group were injected with PBS and FCA only.
^{b}Significance was tested by Chi-square analysis:
**p ≦ 0.01;
*p ≦ 0.05

### EXAMPLE VII

Oral Immunization of Blue Gourami with the Multicomponent Vaccine and Detection of Antibodies

To test for the effectiveness of the multicomponent vaccine against the bacterium Aeromonas hydrophila, 3 groups of 34 gouramis were treated as follows: The first group formed the control and was administered normal feed. The second group was administered vaccine mixed with powdered feed while the third group was administered vaccine emulsified with palm oil and mixed with powdered feed. Fish were similarly boosted after three weeks. Serum was collected from representative fish of each group one week after the booster and assayed for the presence of antibodies using the antibody-antigen agglutination assay. The remaining fish were challenged with live Aeromonas hydrophila.

The antibody-agglutination assay showed that oral administration stimulates the generation of similar titres of antibodies against Aeromonas hydrophila in the immunized fish whether the vaccines were prepared as water-in-oil emulsion or without palm oil.

Effect of vaccine and palm oil emulsion on antibody production in blue gourami.

| Treatment | Antibody titre |
|---|---|
| Control | 1:1 |
| Feed + vaccine (non-emulsion) | 1:4 |
| Feed + vaccine (water-in-oil emulsion) | 1:4 |

During the challenge test, fish that were orally administered the multicomponent vaccine emulsified with water-in-palm oil, showed a higher survival rate than those administered the non-emulsified vaccine as shown in FIG. 1.

### EXAMPLE VIII

Comparison of Results of Oral Immunization and Immunization by Immersion in Protein Cocktail Against Viral Infection

To test for the effectiveness of the multicomponent vaccine and to compare the efficacy of administering the recombinant proteins by the immersion technique, 3 groups of guppies were subjected to different treatments. One group was orally administered the vaccine while the second group was immersed in water containing equivalent dosage of proteins as contained in the oral vaccine. The third group was used as control and given normal feed. Results show that oral vaccination as well as the use of immersion method provided protection against GPV infection as shown in FIG. 2. Neutralizing antibodies were present in oral and immersion-vaccinated fish. However, levels of neutralizing antibodies encountered in orally immunized fish as compared to those of the immersion-vaccinated were not higher.

### Neutralization test using sera obtained from fish surviving challenge with 8 log₁₀TCID₅₀mL⁻¹ virus

Neutralization Index*
Serum dilution

| Route of immunization | Undiluted | ½ | ¼ | ⅛ |
|---|---|---|---|---|
| Oral vaccine | 1.67 | 0.17** | 1.5 | 2 |
| Immersion with proteins | ∞ | ∞ | ∞ | ∞ |

*NI ≦ 1: indicates there is no neutralization NI > 1/NI = ∞: indicates neutralization;.
N: undiluted serum; ½, ¼, ⅛: serially diluted serum with MEM-10
**Cause of cell death undetermined.

In the case of oral immunization, neutralization of virus was observed at all dilutions (NI>1) except dilution of ½ where NI=0.17. Immersion immunization, produces strong neutralization of virus at all dilutions as NI was ∞ signifying that none of the wells which contained serum from control fish were free of CPE.

1. An oral vaccine comprising at least one of recombinant adhesin protein of Aeromonas hydrophila (AHMA), recombinant protein AHMA fragments, and recombinant protein derivatives.

2. The vaccine of claim 1 wherein at least one of the recombinant protein fragments and derivatives is emulsified in water-in-oil emulsion.

3. The vaccine according to claim 2 wherein said emulsifying oil further comprises organic oil.

4. The vaccine according to claim 3 wherein said emulsifying oil further comprises palm oil.

5. The vaccine according to claim 2 wherein the proportion of water and oil in the emulsion is in the ratio of 1:2.

6. The vaccine according to claim 2 wherein the proportion of water and oil in the emulsion is equal.

7. The oral vaccine according to claim 2 mixed with a binding agent.

8. The oral vaccine of claim 7 wherein the binding agent further comprises particulate feed material.

9. The oral vaccine of claim 8 wherein the binding agent further comprises high viscosity carboxymethylcellulose.

10. The oral vaccine of claim 1 comprising an immunologically effective dose of recombinant AHMA protein.

11. The oral vaccine according to claim 1 further comprising recombinant fusion protein from Ichthyophthirius multifiliis (FP).

12. The vaccine of claim 11 wherein the recombinant proteins are emulsified in a water-in-oil emulsion.

13. The vaccine according to claim 12 wherein said emulsifying oil further comprises organic oil.

14. The vaccine according to claim 13 wherein said emulsifying oil further comprises palm oil.

15. The vaccine according to claim 12 wherein the proportion of water and oil in the emulsion is in the ratio of 1:2.

16. The vaccine according to claim 12 wherein the proportion of water and oil in the emulsion is equal.

17. An oral vaccine according to claim 12 mixed with a binding agent.

18. The vaccine according to claim 17 wherein the binding agent further comprises particulate feed.

19. The vaccine according to claim 17 wherein the binding agent further comprises carboxymethylcellulose.

20. The oral vaccine according to claim 11 comprising immunologically effective dose of at least one of the proteins selected from the group consisting of recombinant protein AHMA, recombinant protein AHMA fragments, and recombinant protein derivatives.

21. The oral vaccine according to claim 1 further comprising inactivated viruses elected from a group consisting of guppy reovirus and guppy nervous necrosis virus.

22. The oral vaccine according to claim 1 further comprising bacterial antigens or killed bacteria selected from a group consisting of Shewanella putrefaciens, Pseudomonas fluorescens, Vibrio alginolyticus and Flexibacter columnaris.

23. A method of making an oral vaccine comprising the steps of:
a) separately mixing a predetermined amount of at least one of recombinant protein AHMA, recombinant protein AHMA fragments, and recombinant protein derivatives and whole recombinant protein AHMA, either singly or in combination with at least one antigen selected from the group consisting of recombinant protein FP, guppy reovirus, guppy nervous necrosis virus, Shiwanella putrefaciens, Pseudomonas florescens, Vibrio alginolyticus and Flexinobactor columnaris in a predetermined volume of at least one of water and saline.
b) vigorously mixing a pre-determined volume of organic oil with (a) to form an emulsion.
c) optionally, adding a binding agent to emulsion (b) with gentle stirring to obtain the consistency of a paste, and
d) optionally, adding particulate feed to (c) to obtain a particulate oral vaccine.

24. The method according to claim 23 wherein the organic oil further comprises palm oil.

25. The method according to claim 23 wherein the binding agent further comprises particulate feed.

26. The method according to claim 23 wherein the binding agent further comprises high viscosity carboxymethylcellulose.

27. The vaccine prepared by the method according to claim 48 wherein the computed dosage of recombinant AHMA in the vaccine ranges between 7 μg/g and 150 μg/g body weight of the recipient.

28. The oral vaccine prepared by the method according to claim 48 wherein the amount of recombinant AHMA is between 15 μg/g and 20 μg/g body weight of the recipient.

29. The oral vaccine prepared by the method according to claim 48 wherein the amount of recombinant AHMA is 17 μg/g body weight of the recipient.

30. The vaccine prepared by the method according to claim 48 wherein the computed dosage of recombinant FP in the vaccine ranges between 7 μg/g and 150 μg/g body weight of the recipient.

31. The oral vaccine prepared by the method according to claim 48 wherein the amount of recombinant FP is between 15 μg/g and 20 μg/g body weight of the recipient.

32. The oral vaccine prepared by the method according to claim 48 wherein the amount of recombinant FP is 17 μg/g body weight of the recipient.

33. The vaccine prepared by the method according to claim 48 wherein the computed dosage of at least one of viral proteins and inactivated virus in the vaccine ranges between 10³ and 10⁶ viral particles/g body weight of the recipient.

34. The oral vaccine prepared by the method according to claim 48 wherein the amount of at least one of viral protein and inactivated virus is 10⁵ viral particles/g body weight of the recipient.

35. The vaccine prepared by the method according to claim 48 wherein the computed dosage of at least one of inactivated bacterial and an equivalent amount of bacterial antigens in the vaccine ranges between 10⁵ cfu/g and 10⁷ cfu/g body weight of the recipient.

36. The oral vaccine prepared by the method according to claim 48 wherein the amount of at least one of inactivated bacteria and an equivalent amount of bacterial antigens in the vaccine is 2.5×10⁶ cfu/g body weight of the recipient.

37. A method of treating a species in need of such treatment against aquatic pathogens comprising administering an immunologically effective does of the vaccine according to claim 23.

38. A method according to claim 37, wherein said animal is an aquatic species.

39. A method according to claim 38, wherein the aquatic species is fish.

40. A method according to claim 39, wherein the fish is a guppy.

41. A method according to claim 39, wherein the fish is a blue gourami.

42. A method according to claim 39, wherein the fish is a goldfish.

43. A fish immunized with the oral vaccine of claim 23.

44. An edible product comprising fish immunized or treated with the vaccine according to claim 25.

45. The oral vaccine according to claim 11 further comprising inactivated viruses elected from a group consisting of guppy reovirus and guppy nervous necrosis virus.

46. The oral vaccine according to claim 11 further comprising bacterial antigens or killed bacteria selected from a group consisting of Shewanella putrefaciens, Pseudomonas fluorescens, Vibrio alginolyticus and Flexibacter columnaris.

47. The oral vaccine according to claim 21 further comprising bacterial antigens or killed bacteria selected from a group consisting of Shewanella putrefaciens, Pseudomonas fluorescens, Vibrio alginolyticus and Flexibacter columnaris.

48. An oral vaccine prepared by a method comprising the steps of:
a) separately mixing a predetermined amount of at least one of recombinant adhesin protein of Aeromonas hydrophila (AHMA), recombinant protein AHMA fragments, and recombinant protein derivatives and whole recombinant protein AHMA, either singly or in combination with at least one antigen selected from the group consisting of recombinant fusion protein from Ichthyophthirius multifiliis (FP), guppy reovirus, guppy nervous necrosis virus, Shewanella putrefaciens, Pseudomonas fluorescens, Vibrio alginolyticus and Flexibacter columnaris in a predetermined volume of at least one of water and saline,
b) vigorously mixing a pre-determined volume of organic oil with (a) to form an emulsion,
c) optionally, adding a binding agent to emulsion (b) with gentle stirring to obtain the consistency of a paste, and
d) optionally, adding particulate feed to (c) to obtain a particulate oral vaccine.
